(19) United States
(12) Patent Application Publication
(10) Pub. No.: US 2013/0274567 A1
(43) Pub. Date: Oct. 17, 2013

(54) SENSOR SYSTEM FOR IMPLANTATION INTO A BODY, AND METHOD FOR PRODUCING THE SENSOR SYSTEM

(71) Applicants: Volker Grosser, Rangsdorf (DE); Stephan Guttowski, Berlin (DE); Michael Niedermayer, Glienicke (DE)

(72) Inventors: Volker Grosser, Rangsdorf (DE); Stephan Guttowski, Berlin (DE); Michael Niedermayer, Glienicke (DE)

(73) Assignee: Fraunhofer-Gesellschaft zur Forderung der angewandten Forschung e.V., Munchen (DE)

(21) Appl. No.: 13/879,188

(22) PCT Filed: Oct. 14, 2011
(86) PCT No.: PCT/EP2011/005237

§ 371 Date: Jun. 24, 2013

(30) Foreign Application Priority Data
Oct. 15, 2010 (DE) 10 2010 048 768.6

(51) Int. Cl. A61B 5/07

(52) U.S. Cl. CPC A61B 5/07
USPC 600/301; 264/272.14; 600/302

(57) ABSTRACT

The invention relates to a sensor system for implantation into a body of a living being, comprising a measuring unit for measuring parameters of the body and generating corresponding measurement signals, a transmitting unit for transmitting signals using the measurement signals, a control and analysis unit which is connected to the measuring unit and the transmitting unit in order to process and prepare the measurement signals and control the transmitting unit for transmitting the transmitting signals, an energy storing unit for supplying energy to the units, and an outer casing that at least partly surrounds the measuring unit, the transmitting unit, the analysis unit, and the energy storing unit. The system comprises at least two thinned substrate layers on which the measuring unit, the analysis unit, and the transmitting unit at least partly take the form of circuits, said substrate layers being stacked one above the other and being connected to one another by means of electric vias for the transmission of signals between the substrate layers.

The invention relates to a sensor system for implanting into a body of a living being in accordance with the preamble of the main claim. The invention furthermore relates to a method for the manufacture of such a sensor system.

Such sensor systems serve the measurement of parameters which can characterize a state of the body of the living being such as blood pressure, heart rate or breathing rate or body temperature. The named systems are equipped with transmission units for generating transmission signals such as radio signals or optical signals or acoustic signals by means of which measured values can be transmitted to a remote receiver.

To measure the named parameters, these systems comprise a measuring unit having one or more sensors as well as a control and evaluation unit which is configured as a rule to activate the measuring unit to carry out corresponding measurements, to prepare measured signals generated by the measuring unit and to control the transmission unit to transmit corresponding signals in which, for example, the measured parameters or also other signals derived from the parameters can be encoded.

Furthermore, such systems include an energy storage unit which can frequently be contactlessly charged, for instance via magnetic fields. The named units are finally surrounded by a biocompatible outer skin which is designed as airtight and gastight as a rule.

In addition to applications in the human body, for instance for medical purposes, such sensor systems are being used to an increasing degree also in animal research, in animal husbandry and in animal breeding to control the health state of animals. It is, for example, known to use such sensor systems in fish farming, with these sensor systems being implanted into the fish of a given fish stock in an aquaculture to be able to check the health state of individual fish via the measurement of different vital parameters (movement activity, swim behavior, heart rate and breathing rate, skin resistance and blood pressure). The sensor systems can be read out via a corresponding receiver, for example a radio receiver, and the vital parameters of the individual fish can subsequently be evaluated and checked using a computer. A use in other animals is also conceivable, in particular in mass animal husbandry such as of poultry, pigs and cattle, where the health risk for the animals is particularly high and contagious diseases and parasites can be transmitted particularly easily.

A large problem in the use of such sensor systems is the size of these systems, on the one hand. In large systems, the implanting of the system into the body is frequently difficult and prone to complications as well as cost-intensive and time-intensive. Furthermore, the risk of complications triggered by the implant also increases as the size of an implanted system increases.

It is therefore the object of the present invention to propose a sensor system which alleviates the named problems, that is, is particularly small and can thus be easily implanted into the body of a living being and also causes complications as rarely as possible in the implanted state. The sensor system should therefore, on the one hand, be as small as possible, but, on the other hand, furthermore be suitable for the secure and precise measurement of as many parameters of the body as possible and should allow a corresponding signal transmission to a remote receiver. Furthermore, a method of manufacturing such a system which is as simple and as inexpensive as possible is to be proposed.

This object is achieved in accordance with the invention by a sensor system and a manufacturing process in accordance with the independent claims. Further developments and embodiments are the subject of the independent claims.

Accordingly, a sensor system in accordance with the invention for implanting into a body of a living being comprises a measuring unit for measuring parameters of the body and for generating corresponding measured signals, a transmission unit for transmitting signals using the measured signals and a control and evaluation unit connected to the measuring unit and to the transmission unit for treating and processing the measured signals and controlling the transmission unit for transmitting the transmission signals. In this respect, the treatment and/or the processing of the measured signals can include a digitizing of analog measured signals, a signal amplification and/or a signal filtering, for instance for noise suppression.

The system moreover has an energy storage unit for the energy supply of the units and an outer skin which at least partly surrounds the measuring unit, the transmission unit, the evaluation unit and the energy storage unit.

It is decisive for the solution in accordance with the invention of the above-worded object that the system comprises at least two thinned substrate layers on which the measuring unit, the evaluation unit and the transmission unit are at least partly implemented as switching circuits, i.e. switching circuits of the measuring unit, of the evaluation unit and of the transmission unit are integrated on the substrate layers. A switching circuit should here be understood as a unit of an electrical or electronic circuit which is configured for satisfying a defined function and comprises corresponding functional components or devices for this purpose such as sensors for measuring measurement values such as (physiological) parameters of a body, devices for evaluating corresponding measured signals, devices for generating control signals for controlling other functional devices, devices for transmitting and/or receiving signals (such as antennas or photosensitive elements) at/of external transmitters or target receivers. In this respect, the substrate layers are stacked over one another and are connected to one another by electrical vias for the signal transmission between the substrate layers so that the switching circuits of the individual units of the sensor system are connected to one another to form a total switching circuit of the sensor system. Thinned substrate layers and substrate layers stacked over one another which are connected to one another by vias (through-silicon vias, TSV) are known from other areas of the art.

As a rule, SMT chips are respectively used in sensor systems of this category for manufacturing the measuring unit, the evaluation unit and the transmission unit. In contrast, these units in the sensor system in accordance with the invention are, as described above, at least partly integrated on thinned, stacked substrate layers. A substantial advantage of stack technology of thinned substrate layers (wafers) is now that a plurality of substrate layers (including the switching circuits integrated on these substrate layers) are layered in a stack which takes up a particularly small construction space. Substrate layers having thicknesses in a range between 5 μm and 25 μm are preferably used. Typical stack heights amount to about 0.1 mm to 0.2 mm or less in dependence on the thickness and number of the stacked substrate layers. Typical lengths and widths of the stack amount to around 3 mm, preferably less.

The construction space required by such a stack can thus be less than a single conventional SMT chip. The sensor system in accordance with the invention can therefore be manufactured in a much more compact manner with smaller overall dimensions by the use of thinned, stacked substrate layers than a corresponding conventional sensor system on the basis of SMT chips. The sensor system in accordance with the invention typically has external dimensions of only 15 mm×4 mm×3 mm (length×width×thickness) or less and is thus much smaller in size than conventional sensor systems having comparable functionality. They typically have external dimensions such as 40 mm×20 mm×8 mm (length×width×thickness) or are even larger.

The named substrate layers and the energy storage unit are preferably completely surrounded by the outer skin. Parts of the measuring unit which are not arranged on one of the substrate layers such as a possibly provided pressure sensor or possibly provided electrodes for measuring skin potentials can also be arranged at least party at or outside the outer skin such as will be described further below. It is an important object of the outer skin to mechanically stabilize the substrate layers, the energy storage unit, possibly present wires and cables for energy transmission and/or signal transmission and to protect them from forces acting from the outside.

Provision is made in a further development that the transmission unit is arranged on a first one of the substrate layers and that an intermediate layer which screens the transmission signals of the transmission unit is provided between the first substrate layer and a further one of the substrate layers. It can be ensured in this manner that signals emanating from the transmission unit do not interfere with or negatively influence the switching circuits of the other substrate layers. This is in particular decisive in the case of radio signals which could interfere with electronic processes within the named switching circuits and thus result in interference without such an intermediate layer. In particular a metallic intermediate layer which can be applied, for example, in the form of a coating to a lower side of the first substrate layer bearing the transmission unit is suitable for screening radio signals. The intermediate layer is preferably produced from a metallic material such as copper and has a thickness in a range between 5 μm and 25 μm.

In a further development of the sensor system which is characterized by a particularly easy implantability, the outer skin is elongated and flat. This then preferably has a length which is larger by a factor X than a width and a height of the outer skin, with the factor X lying in a range between 2 and 20, preferably in a range between 3 and 10. The outer skin is preferably manufactured from a biocompatible potting material such as silicone or from a biocompatible thermoplastic material. The manufacture is possible in a particularly simple and inexpensive manner in this way. A particularly reliable mechanical stabilization of the substrate layers, of the energy storage unit and of the remaining components of the systems (such as sensors, wires, cables) can be achieved by use of such a potting material for the outer skin, see above, since all the components can be at least partly surrounded by the potting material and can thus be supported and protected from deformations, displacements or twisting.

Furthermore, practically any desired shapes for the outer skin can be realized by use of a suitable mold in the manufacture. Outer skins in the form of preferably prolate ellipsoids or elongated cylinders with rounded edges are particularly well-suited, for example. Overall, elongated and preferably convex outer skins are characterized by an easy implantability and by particularly good compatibility in the implanted state. Outer skins shaped in this manner can in particular be particularly easily pushed underneath the skin of a person or of an animal. The shape of the outer skin is preferably tapered at at least one side so that a better implantability is achieved.

Provision is made in a further embodiment that the measuring unit includes a temperature sensor for measuring a body temperature. This temperature sensor can be arranged directly on one of the thinned substrate layers. The temperature sensor is preferably designed as a high-ohmic resistance sensor. Correlation software is preferably installed in the associated evaluation circuit which (after a preceding calibration) carries out a conversion of the measured temperature to an internal body temperature.

It is additionally possible that the measuring unit comprises an accelerometer for measuring an acceleration of the body by means of which a conclusion can be drawn on a movement behavior of the living being. With fish, for example, it is thus possible to observe their swimming behavior with the aid of the accelerometer. This swimming behavior can comprise characteristic features such as jerky swimming or movement inertia on a disease infestation of a fish. The accelerometer, as also the temperature sensor, is preferably arranged directly on one of the thinned substrate layers for a particularly compact construction of the system. The accelerometer can be equipped with micromechanical comb structures which are oriented orthogonally to one another to be able to determine the acceleration in the three spatial dimensions.

In addition or alternatively to the above-named sensors, the measuring unit can comprise at least one pressure sensor which is preferably arranged at an outer side of the outer skin and with which a pressure in the body can be measured. In this respect, the at least one pressure sensor is connected to the switching circuit of the measuring unit arranged on one of the thinned substrate layers via one or more lines, e.g. a wire line, via which its measured pressure signals can be transmitted for further processing to this switching circuit. In a particularly preferred embodiment, a first pressure sensor and a second pressure sensor are provided which are arranged at mutually opposite sides of the outer skin. The precision of the pressure measurement is increased in this manner. The pressure sensors can in turn be present as microchips, for example having a micromechanical membrane, preferably silicon-based. In a further development, the evaluation unit is configured for determining a frequency spectrum of measured signals generated by the at least one pressure sensor and for determining a blood pressure, an environmental pressure, a heart rate and/or a breathing rate of the body from the frequency spectrum. In this respect, the heart rate is from higher frequency portions, the breathing rate from lower portions and the diving depth or an environmental pressure from quasi-static portions.

In addition or alternatively to the already named sensors, the measuring unit of the system can comprise at least two electrodes which are arranged on an outer surface of the outer skin and which serve the measurement of a skin potential and/or of a (inner) skin resistance. In this respect, the at least two electrodes are connected via a line to the switching circuit of the measuring unit arranged on one of the thinned substrate layers for the signal transmission between the at least two electrodes and this switching circuit, for example to a respective at least one wire or cable. At least two of these electrodes preferably run around the outer skin in ring form. In the event that the outer skin is elongated, as described above, these electrodes preferably run around the outer skin transversely to a longitudinal axis of the outer skin.

In this manner, a contact can particularly easily be established between these electrodes and the skin of the living being since this contact can exist independently of an angle of rotation of the system about the named longitudinal axis. A corresponding measurement is carried out by a resistance measurement between these electrodes. (Pure) gold or another metal, which is as precious as possible, is used as the electrode material; the thicknesses of the electrodes preferably lie in a range between 0.01 mm to 2 mm, preferably between 0.01 mm and 0.5 mm, and preferably have spacings between one another between 2 mm and 5 mm.

Provision is made in a further development that the energy storage unit comprises at least one first and one second electrical energy store, with the first energy store having a predefined negative anode potential and the second energy store having a predefined positive cathode potential. This allows a particularly reliable and precise measurements of the skin potential even when it varies over time and even when it changes its sign. The skin potential at specific excitation states (in fish, for instance) may thus change its sign (“reverse polarity”), for example. In such a case, a second (reversed) reversed voltage is required for the measurement. A preferred value for the anode potential of the first energy store amounts to about −3 V and a preferred value for the cathode potential of the second energy store amounts to about +3 V.

Plate-like or planar batteries or storage batteries are preferred as energy stores since they allow a particularly compact design of the sensor system, particularly when these energy stores are oriented substantially (i.e. within the framework of production tolerances) parallel to one another, i.e. are arranged areally above one another. In addition to lithium ion batteries, in particular also film batteries can be considered such as are known, for example, from the documents DE 10 2007 031 477 A1 as well as DE 103 46 310 A1. They are characterized by a particularly high energy density and can additionally be shaped flexibly and are in this manner particularly well-suited for a small construction space.

A particularly compact construction manner of the sensor system can be achieved when an end piece of one of the substrate layers which is connected to the contacts of the two energy stores is arranged between the at least one first and second energy stores. Provision can be made in this respect that an upper side of this end piece contacts the first energy store and a lower side contacts the second energy store. Furthermore, a voltage adaptation circuit can be integrated on the named substrate layer, preferably on the named end piece of this substrate layer, and delivers the supply voltage for the measuring unit, for the evaluation unit and/or for the transmission unit. The system can be equipped with a switch, for example with a magnetic switch such as a so-called reed contact or a reed relay which (in the first use) is “switched on” contactlessly and thus sets the system into the operating state. In the switched-off state, the sensor preferably does not consume any energy.

In a further development, the energy storage unit comprises a coil for the contactless charging of the energy storage unit, e.g. by means of magnetic energy. In this respect, the coil can be arranged as a conductor track on one or more of the substrate layers or as a wire coil which runs around the stacked substrate layers and the energy storage unit. The energy storage unit can be recharged using such a coil by means of a remote charging unit which irradiates magnetic energy. In this respect, a corresponding charging process can also be carried out in the implanted state. A maximum distance between the system and the charging unit which can be achieved for the charging process in this respect substantially depends on a transmission power of the charging unit and a sensitivity (inductance) of the coil. This maximum distance can amount to several meters. It is in particular also possible to charge systems which are implanted in fish while they are swimming in water, for example in an aquaculture or in free waters.

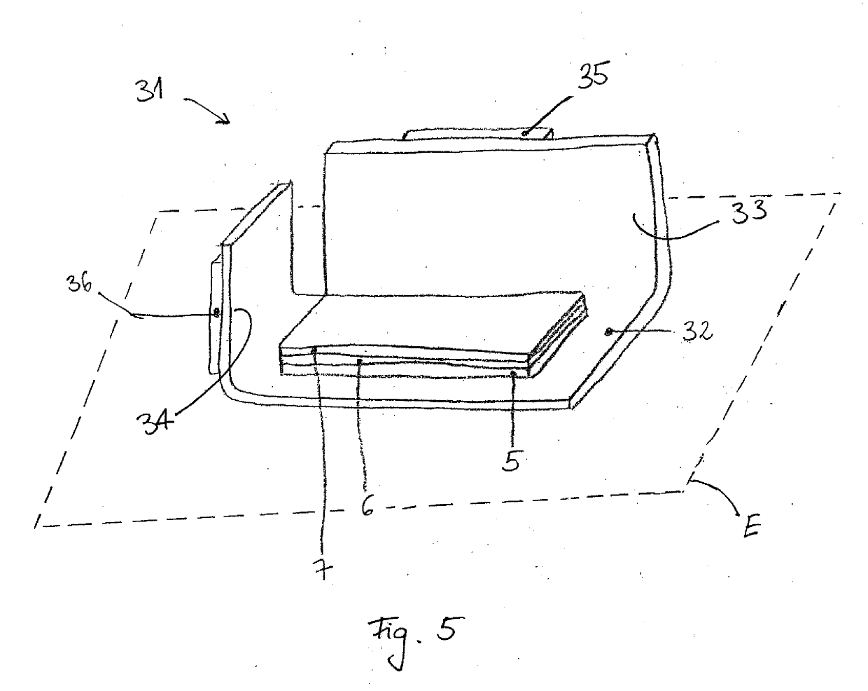

The following advantages can furthermore be achieved with the invention:
Cost reduction by minimizing the material used (i.e. mm² of silicon area)
Thinned substrate layers, for instance comprising silicon, are flexible and can be brought into a desired or required position for the sensor (see following description and FIG. 5) by bending.
Since each (part) switching circuit can be located on a separate substrate, a single testing and singling out of defective switching circuits can take place at an early stage of the production.
The (part) switching circuits can be selected and mounted in dependence on the application both within the framework of a mass production and also in a user-specific (single) production.
The extreme miniaturization allows the implanting of the sensor also in young animals in which the implanting of larger sensor systems typically results in a high mortality rate.
A flexible arrangement of the active sensor surfaces in all spatial directions such as is required by the measurement value to be detected by the sensor (optionally in dependence on the direction) can be achieved by utilizing the flexibility of the substrate surfaces. Functional components of transmission and reception units (such as antennas) can in this manner also be directly oriented, for instance to increase sensitivity or to reduce a required transmission power, see following description.
In addition, by the arrangement of the sensors of the measuring unit on one of the substrates layers, signal conductors for transmitting measured signals can also be integrated into this substrate layer. Separate line element such as flexible lines can thereby be saved.

The present application is additionally directed to a stack of two or more thinned substrate layers, with at least one of these substrate layers being bent so that a part region or several part regions of this at least one substrate layer projects out of a common main plane of the stacked substrate layers. The common main plane of the stacked substrate layers is in this respect defined so that the main plane extends parallel to the stacked substrate layers where the substrate layers are stacked in planar form over one another and are oriented in parallel with one another.

On a permanent shaping of the thinned substrate layers by bending, the fact is utilized that the substrate layers are even flexible, due to their small thickness, when they are produced from a relatively brittle material such as silicon, for instance. In this respect, an achievable radius of curvature of the bent at least one substrate layer typically depends on a thickness of this substrate layer (and on the material of the substrate layer). The thinner the respective substrate layer, the smaller curvature radii can generally be achieved. For example, with substrate layers comprising silicon having a thickness of approximately 10 μm, radii of curvature of around 1 mm or less can be achieved.

To maintain or fix a bent, thinned substrate layer in its form, a corresponding holding element or several holding elements can be provided with which the position of the at least one part region of the respective substrate layer projecting out of the main plane of the stack is stabilized and fixed. Such a holding element can, for example, be given by a region of an outer skin or of an encapsulation of the stack. Provision can also be made that the stack is fastened to a preferably rigid support which additionally has corresponding holding elements. Such a holding element can, for example, also be designed as a clamp, a spigot, a projection, a recess or a passage opening of such a support or of such an outer skin or of such an encapsulation.

One or more functional components/devices such as electronic, photoelectrical, micromechanical or microfluidic components, in particular sensors for detecting measurement values or functional devices/components of transmission and/or reception units for transmitting and/or receiving signals can now be placed on the named at least one part region of such a bent substrate layer which therefore projects out of the main plane of the stack of substrate layers. In principle, all sufficiently greatly miniaturized sensors such as sensors for measuring a pressure (for instance of blood), a temperature, an acceleration, a radiation intensity (for instance of light, also with spectral resolution), a material concentrations (for instance of sugar or insulin, for example in the blood), a pH (for example of blood), a moisture, an electrical current, an electrical voltage, an electrical field or a magnetic field (for instance using a Hall probe) can be considered as sensors. For example, acoustic (for instance for ultrasonic frequencies), electrical, magnetic, electromagnetic (for example antennas for radio frequencies, for instance 2.4 GHz), optical and thermal components or devices can be considered as functional components/devices of the transmission and reception units.

Since this at least one part region projects from the named main plane and includes a predefined angle with the main plane, the components arranged on the part region have a predefined spatial arrangement and orientation to the main plane. In the event that the functional component arranged on the respective part region is a sensor, measurement values depending on direction, angle and/or position can thereby be directly detected and, for example, the dependence on direction, angle and/or position of these measurement values can also be resolved. For example, two or more sensors can be arranged on different part regions of one or more substrate layers of the stack, with these part regions including fixedly predefined angles with the main plane and also with one another. For example, a radiation intensity or an electrical or magnetic field can be measured simultaneously in different directions using such an arrangement. In this manner, the propagation of pressure waves (in air or in liquids) can also be resolved in space and in time and in this manner, for example, with reference to time of flight differences, a source of the pressure waves can be localized or also signal portions which are associated with a specific source such as with a heart, can be filtered, for instance to subsequently subject them to a further analysis such as the above-described frequency analysis. The control and evaluation unit can be correspondingly configured for such analyses and evaluations of the measured signals. An advantage of an evaluation of measured signals by means of the control and evaluation unit of the sensor system which is a far-reaching as possible is that in this manner a considerable data reduction is often achieved starting from the measured signals. Only still relatively few data amounts usually have to be transmitted to external target receivers subsequently to such an evaluation, whereby frequently a considerable saving in transmission energy consumed by the transmission unit can be achieved.

If the functional component which is arranged on the part region projecting out of the main plane of the stack is a component of a reception unit (such as an RF antenna or a photosensitive sensor), signals which—emanating from a specific signal direction—are incident on the reception unit are received particularly well if the reception unit is oriented so that the reception unit is particularly sensitive for signals from this signal direction. Such an optimized orientation of the reception unit can be achieved by a corresponding orientation of the part region on which the reception unit is arranged, that is by a corresponding bending of the substrate layer of this part region until the named (ideal) orientation of the reception unit is achieved.

If the functional component which is arranged on the part region projecting from the main plane of the stack is a component of a transmission unit (such as an RF antenna or an optical transmitter), signals which should be transmitted in a specific signal direction (for instance because a target receiver for the transmitted signals is located in this direction) can be specifically directed and thus can be transmitted in a particularly energy-saving manner if the transmission unit itself is oriented so that its specific transmission power is particularly large in this signal direction. Such an optimized orientation of the transmission unit can in turn be achieved by a corresponding orientation of the part region on which the transmission unit is arranged, that is by a corresponding bending of the substrate layer of this part region until the named (ideal) orientation of the transmission unit is achieved.

Provision can thus be made, for example, that the at least one part region includes an angle with the common main plane of the substrate layers of at least 20°, of at least 30° or of at least 45°. This angle amounts to (around) 90° in a particularly preferred embodiment. Provision can also be made that two part regions which project out of the main plane include an angle of at least 20°, of at least 30° or of at least 45°. The angle between two part regions can also amount to (about) 90°.

The substrate layers of the sensor system proposed here can, as described above, be bent and can, likewise as described above, have functional devices such as the transmission unit, a reception unit and/or sensors of the measuring unit on the named part regions to achieve the described advantages and suitabilities. It must, however, be stressed at this point that those stacks of thinned substrate layers which are bent such that at least one part region of at least one of these substrate layers projects from the named main plane of the stack can also be used for other purposes and are not restricted to sensor systems of the kind proposed here.

As a rule, a system comprising such a stack of thinned and partly bent substrate layers is, however, likewise a measuring unit for measuring (for example physical or chemical) measurement values and for generating corresponding measured signals, a transmission unit for transmitting signals using the measured signals and/or a control and evaluation unit connected to the measuring unit and to the transmission unit for processing the measured signals and controlling the transmission unit for the transmission of the transmission signals. The system can furthermore comprise an energy storage unit for the energy supply of the units. In this respect, switching circuits of the measuring unit, of the evaluation unit and of the transmission unit can be integrated on the at least two thinned substrate layers of the system, with the substrate layers being stacked over one another and being connected to one another by electrical vias for the signal transfer between the substrate layers.

The method in accordance with the invention for manufacturing a sensor system of the kind proposed here accordingly comprises the following steps:
integrating switching circuits of the measuring unit, of the evaluation and control unit and of the transmission unit on thinned substrate layers;
stacking the substrate layers:
connecting the substrate layers by means of vias;
connecting the energy storage unit to the measuring unit, to the evaluation and control unit and/or to the transmission unit, for example by at least one line such as a wire or cable or by direct contacting (e.g. by soldering, brazing the energy storage unit to the substrate);
fixing the measuring unit, the evaluation and control unit, the transmission unit and/or the energy storage unit within a mold, for example using mold lugs and/or at least one clamp; and
casting the mold having a preferably biocompatible potting material, for example with silicone or a biocompatible thermoplastic material.

Different orders of the named steps are possible in this respect. For example, the measuring unit, the evaluation and control unit and/or the transmission unit can thus first be connected to the energy storage unit and the units can subsequently be fixed in the mold. It is, however, also possible to fix the measuring unit, the evaluation and control unit and/or the transmission unit and the energy storage unit in the mold and subsequently to connect these units to one another.

A further development of this method moreover includes at least one of the following further steps:
connecting at least two, preferably three, and preferably ring-shaped electrodes for measuring a skin resistance to the switching circuit of the measuring unit on one of the substrate layers, for example by at least one line, e.g. a wire, and fixing the electrodes at an inner surface of the mold;
connecting at least one pressure sensor to the switching circuit of the measuring unit on one of the substrate layers, for example by at least one line, e.g. a wire, and fixing the at least one pressure sensor to the inner surface of the mold;
placing at least one functional component such as a sensor of the measuring unit, a component of the transmission unit and/or a reception unit for signals of an external transmitter, on at least one part region of at least one substrate layer, with the at least one part region either already projecting out of a common main plane of the stacked substrate layers or being bent out in a subsequent step by bending this substrate layer;
bending at least one of the substrate layers so that the at least one part region of this at least one substrate layer projects from a common main plane of the stacked substrate layers;
fixing the at least one part region of the bent substrate layer(s) by means of holding elements and/or by means of the mold.

In this respect, the bending of the at least one substrate layer can be carried out before or after the integration or placing of the named switching circuits, functional components/devices, sensors, transmission units and/or reception units on the respective substrate layer as well as before or after the stacking of the substrate layers. The integration or placing of the switching circuits, of the functional components/devices, of the sensors, of the transmission and/or reception unit is preferably carried out before substrate layers are bent. Provision can be made that, after bending the at least one substrate layer, the stack of the substrate layers is inserted into the mold so that the at least one part region projecting out of the common main plane of the stack is stabilized by the mold or by holding elements (of the mold). The named part regions can be permanently stabilized by a subsequent casting of the mold with the potting material, in particular also after removing the system from the mold.

It is possible to carry out the named additional method steps in any desired orders. In the event that two pressure sensors are provided, they are fixed at two mutually opposite sides of the inner surface of the mold.

Generally, a method for manufacturing a stack of thinned and partly bent substrate layers, as described above, can comprise the following steps:
integrating switching circuits of the measuring unit, of the evaluation and control unit, of the transmission and/or reception unit on thinned substrate layers;
stacking the substrate layers;
connecting the substrate layers by means of vias;
placing at least one functional component/device of the measuring unit (e.g. of a sensor), a functional component/device of a transmission unit and/or reception unit (e.g. of an antenna of these units) for signals of external transmitters or receivers respectively on at least one part region of at least one thinned substrate layer of the stack;
bending at least one of the substrate layers so that at least one of the part regions of this at least one substrate layer projects from a common main plane of the stacked substrate layers;
fixing the at least one part region by means of at least one holding element and/or within a mold;
casting the mold with a potting compound.

In accordance with the proposes method for manufacturing a sensor system, the order of the steps, in particular of the first three named steps, can also be swapped around here.

The invention will be explained in more detail in the following with respect to two specific embodiments shown in the drawings.

There are shown:

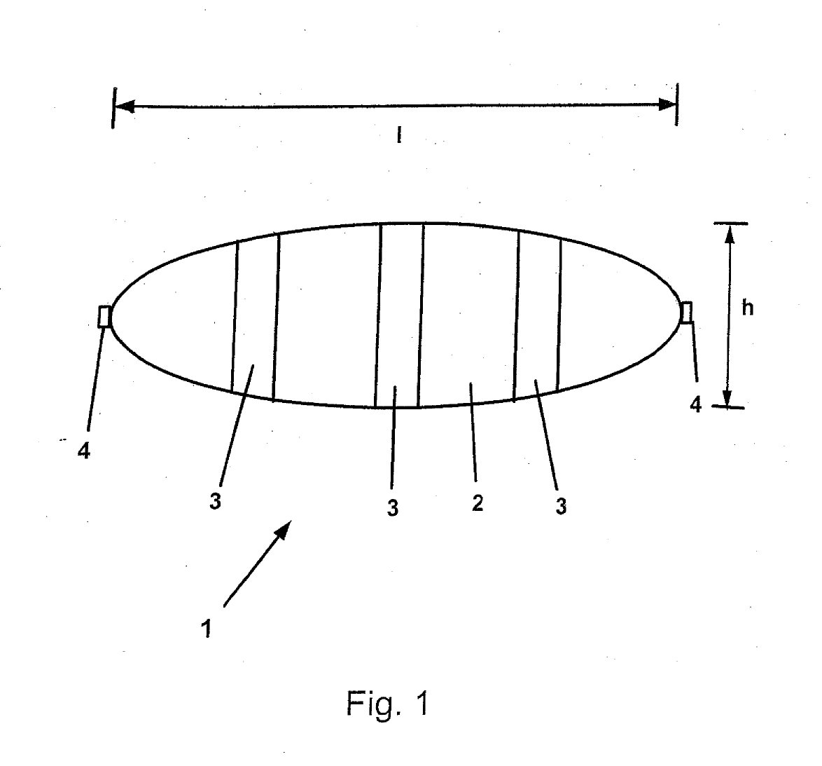

FIG. 1 a sensor system of the kind proposed here;

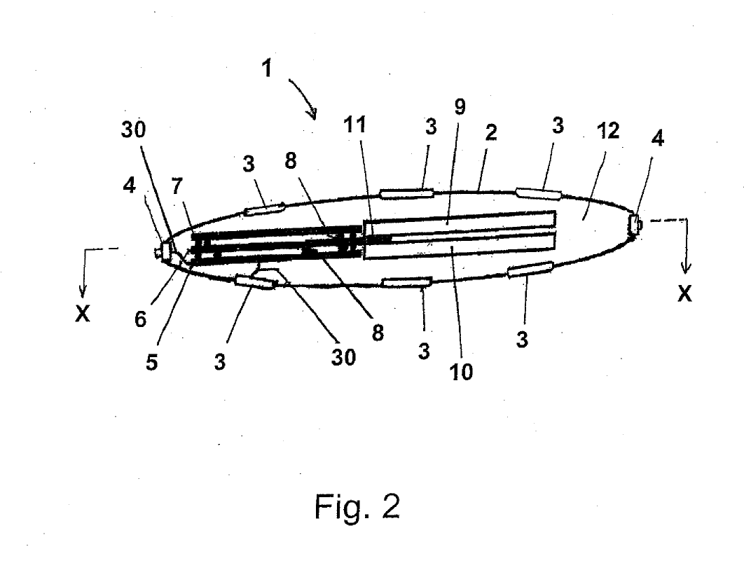

FIG. 2 a longitudinal section through the sensor system shown in FIG. 1;

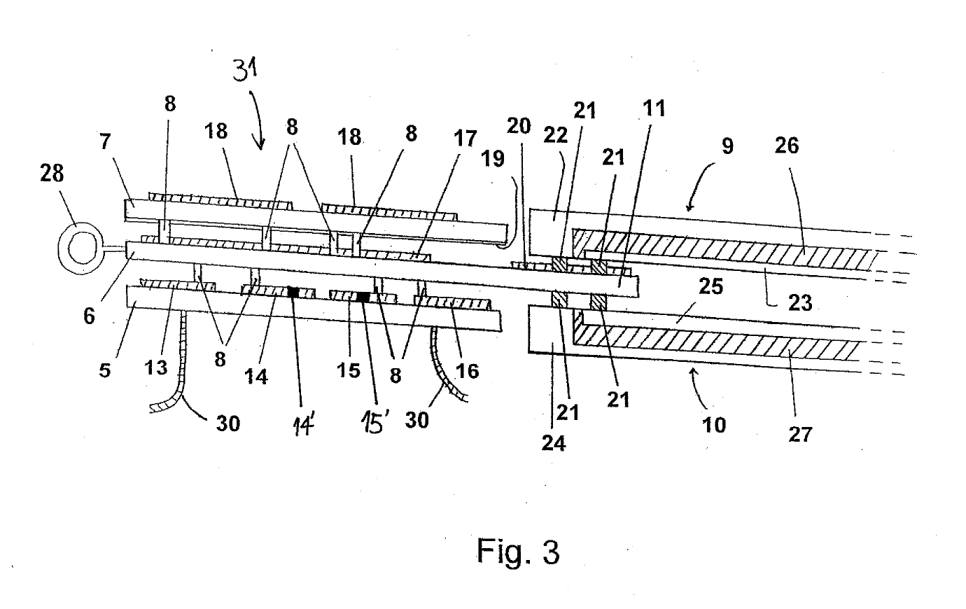

FIG. 3 an enlarged detail of the representation shown in FIG. 2;

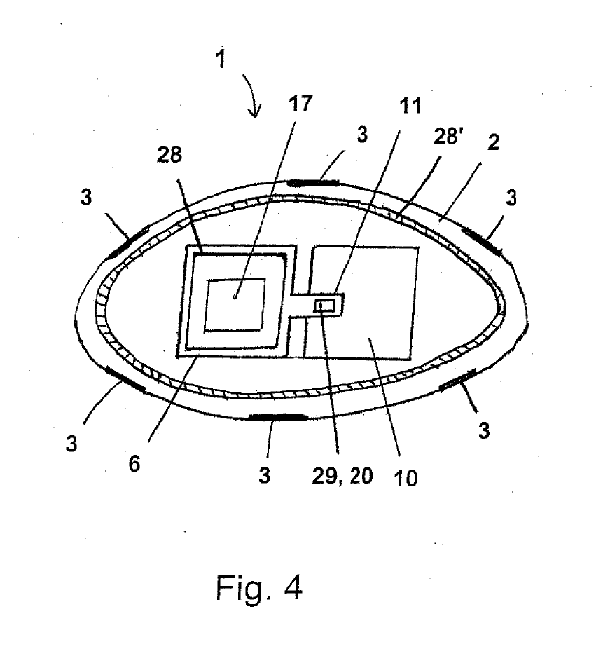

FIG. 4 a further sensor system of the kind proposed here in a longitudinal section;

FIG. 5 a stack of thinned substrate layers of the kind proposed here; and

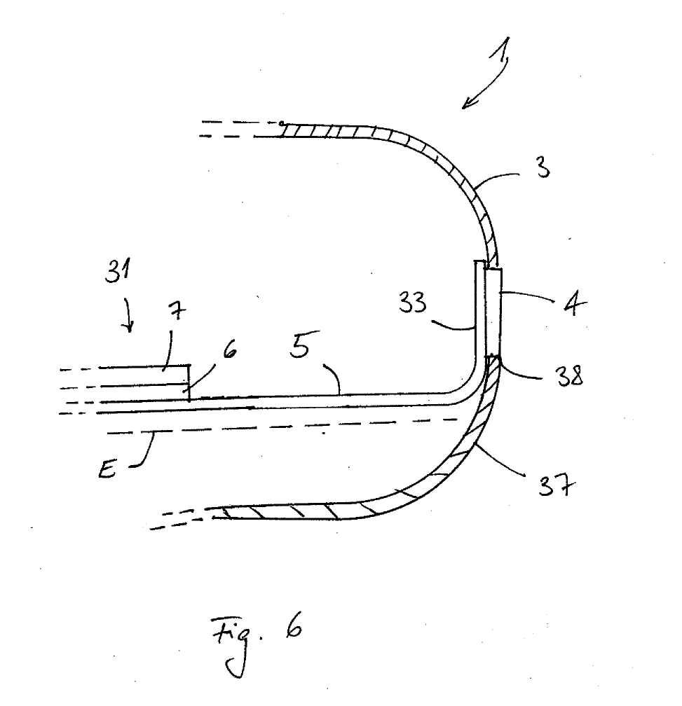

FIG. 6 a stack of thinned substrate layers of the kind proposed here.

In this respect, recurring reference numerals designate the same features.

The sensor system 1 shown schematically in FIG. 1 represents a preferred embodiment of the proposed invention which is suitable for implanting into a body of a living being, for example for implanting into a fish. The sensor system 1 comprises an elongated outer skin 1 which is manufactured from a biocompatible potting material which is given by silicone in this example. A length 1 of the outer skin amounts to around 15 mm; a height h to around 4 mm and a depth (measured perpendicular to the plane of the drawing) to around 3 mm. The outer skin in this respect has an ellipsoid-like form and can thus be particularly easily implanted beneath the skin of a fish.

The sensor system comprises three electrodes 3 for the measurement of an inner skin resistance and a skin potential, said electrodes being produced from a biocompatible metal material, running around the outer skin in ring form at an outer side and transversely to the longitudinal axis (along which the length 1 is entered) of the system 1 and being arranged substantially concentric. The system 1 furthermore has two pressure sensors 4 which are arranged at two mutually opposite sides of the outer side of the outer skin 2 for measuring blood pressure, a heart rate and breathing rate and a diving depth of a fish.

A longitudinal section through the sensor system 1 illustrated in FIG. 1 is shown schematically in FIG. 2. The sensor system 1 comprises a first thinned substrate layer 5 on which a switching circuit of a measuring unit is integrated; a second thinned substrate layer 6 on which a control and evaluation unit is integrated; and a third thinned substrate layer on which a switching circuit of a transmission unit is integrated. The named substrate layers 5, 6 and 7, which are produced from silicon, are stacked over one another to form a stack and are connected to one another by means of vias 8, so-called through-silicon vias (TSVs), for a signal transmission between the switching circuits integrated on the substrate layers, cf. FIG. 3.

The thinned substrate layers each have a thickness of 5 μm to 25 μm so that the stack formed by the substrate layers has a total height of only 100 μm to 200 μm.

The pressure sensors 4 and the electrodes 3 are each connected via electrical connectors 30 such as electrical lines or wires (only drawn in part) to the switching circuit of the measuring unit integrated on the first substrate layer 5 for transmitting signals to this switching circuit.

The third substrate layer 7 with the transmission unit which comprises a radio element for transmitting radio signals at a 2.4 GHz frequency is arranged as the uppermost of the three substrate layers 5, 6 and 7.

The second substrate layer 6 arranged between the first and third substrate layers 5 and 7 has an end piece 11 which protrudes opposite the first substrate layer 5 and the third substrate layer 7 and which is arranged between a first and a second energy store 9 and 10. These energy stores 9 and 10 are designed as film storage batteries and together form an energy storage unit for the energy supply of the named components of the system 1.

An inner space 12 of the system is completely filled by the potting material of the outer skin 2. In other words, the outer skin 2 projects up to the stack of the thinned substrate layers 5, 6 and 7 and up to the energy storage unit 9, 10 and supports these components of the system 1. In this manner, these components are mechanically stabilized and protected from external forces.

An enlarged section of the longitudinal section shown in FIG. 2 is shown schematically in FIG. 3. A total switching circuit of the measuring unit is integrated on the first thinned substrate layer 5 and comprises a first switching circuit 13 which belongs to the pressure measurement and which is connected to the pressure sensors 4 via electrical connectors 30 such as wires (cf. FIGS. 1 and 2) and comprises a second switching circuit 14 which belongs to the temperature measurement and into which a temperature sensor 14′ is integrated, for example in the form of a temperature-dependent resistance element, and also comprises a third switching circuit 15 which belongs to the acceleration measurement and into which an accelerometer 15′ is integrated, for example in the form of micromechanical comb structures oriented orthogonal to one another, and comprises a fourth switching circuit 16 which belongs to the skin potential measurement and skin resistance measurement and which is connected to the electrodes 3 via electrical connectors 30.

The four named switching circuits 13, 14, 15, 16 which form parts of the total switching circuit of the measuring unit are configured for amplifying measured signals of the respective sensors (i.e. of the pressures sensors 4, of the electrodes 3, of the temperature sensor and of the accelerometer) and for forwarding these measured signals via vias 8 to the evaluation and control unit which is implemented as a switching circuit 17 on the second substrate layer 6. The evaluation and control unit 17 is configured to activate the above-named switching circuits 13, 15, 15 and 16 of the measuring unit to carry out measurements and to evaluate and digitize the measured signals thereupon received.

The evaluation and control unit 17 is in particular configured to subject the measured signals belonging to the pressure measurement to a frequency analysis and to calculate a heart rate and a breathing rate, a blood pressure and a diving depth of the fish. The evaluation and control unit 17 is furthermore configured to calculate a body temperature of the fish from the measured signals belonging to the temperature measurement and to calculate a swimming behavior from the measured signals belonging to the acceleration measurement and to recognize specific movement features (such as jerky swimming and movement inertia). Finally, the evaluation and control unit 17 is configured to calculate a skin potential and a skin resistance from the measured signals belonging to the skin potential and skin resistance measurement.

The evaluation and control units 17 is additionally configured to transfer the parameters determined in this manner (body temperature, skin potential and skin resistance measurement, movement features, swimming behavior, heart rate and breathing rate, blood pressure and diving depth) to a switching circuit of a transmission unit 18 integrated on the third thinned substrate layer 7 by means of the vias 8. The radio element of the system is integrated into this switching circuit 18. This switching circuit 18 is configured to generate radio signals by the radio element in which radio signals the named parameters are encoded.

A metallic coating 19 is applied to a lower side of the third substrate layer 7. This coating 19 represents an intermediate layer 19 for screening the first and second substrate layers 5 and 6 arranged beneath the third substrate layer 7 with respect to radio signals of the radio element 18.

A circuit 20 which comprises a recharging circuit 20 and also a voltage adaptation circuit 20 is integrated on the end piece 11. This switching circuit is via contacts 21 to an anode 22 of the first energy store 9, to a cathode 23 of the first energy store 9, to an anode 24 of the second energy store 10 and to a cathode 25 of the second energy store 10.

An electrolyte 26 is arranged between the anode 22 of the first energy store 9 and the cathode 23 of the first energy store 9. An anode potential of the first energy store 9 amounts to approximately −3 volts; a cathode potential of the first energy store to approximately 0 volts.

An electrolyte 27 is arranged between the anode 24 of the second energy store 10 and the cathode 25 of the second energy store 10. An anode potential of the second energy store 10 amounts to approximately 0 volts; and a cathode potential of the second energy store to approximately 3 volts.

The recharging circuit 20 is configured to recharge the two energy stores 9 and 12. The recharging circuit 20 can be activated by an external charge unit (not shown). For this purpose, the recharging circuit is connected to a coil 28 arranged at an outer margin of the second substrate layer 6 for transmitting electrical energy from the coil 28 to the recharging circuit 20. As soon as such an energy transfer takes place, the recharging circuit 20 is activated; the received electrical energy is converted into a suitable charging current to charge the two energy stores 9 and 10. The coil 28 is configured to receive magnetic energy which can be transmitted by a remote charging unit (not shown).

To manufacture the sensor system 1 described with reference to FIGS. 1, 2 and 3, the named circuits 13, 14, 15, 16, 17, 18, 20 are integrated in a first step on the three named substrate layers 5, 6, 7 and the metallic intermediate layer 19 is applied to a lower side of the third substrate layer 7 which supports the transmission unit 18. Subsequently, these substrate layers are stacked over one another and are connected to one another by vias 8. Furthermore, the coil 28 is arranged at an outer margin of the second substrate layer 6 and is connected to the voltage adaptation circuit 20 and to the recharging circuit 20 which is arranged on the end piece 11 of the second substrate layer for the energy transfer. The two energy stores 9 and 10 are equally connected to the voltage adaptation circuit 20 and to the recharging circuit 20 via the contacts 21 for the energy transfer.

The stacked substrate layers 5, 6 and 7 are fixed together with the energy stores 9 and 10 in a mold by means of fixing elements such as clamps or abutments. The two pressure sensors 4 and the three electrodes 3 are equally fixed at an inner surface of the mold, with the two pressure sensors being arranged at two mutually opposite points of the mold. Subsequently, the pressure sensors 4 are connected to the circuit 13 and the electrodes to the circuit 16 of the measuring unit by means of electrical connectors 30 such as wires. The mold is subsequently filled with a biocompatible potting compound, silicone in this case, for manufacturing the outer skin 2.

A further sensor systems of the kind proposed here is shown schematically in a longitudinal section in FIG. 4. This embodiment only differs from the embodiment shown in FIG. 1 by the arrangement of two coils 28, 28′ for charging the energy stores 9, 10 and a reed contact 29 (reed relay) for switching on the sensor system. All other features are the same as those of the sensor system shown in FIG. 1 and have the same reference numerals. The sectional plane extends along the sectional plane X shown in FIG. 2, that is in parallel with and directly above the second substrate layer 6 when looking onto this substrate layer 6.

The first of these two coils 28 is designed as a conductor track on the second substrate layer 6, but could just as easily be arranged on one of the other substrate layers. This conductor track runs along an outer contour of this substrate layer, i.e. as close as possible to the outer edges of the substrate layer and as parallel as possible therewith to achieve an inductivity which is as high as possible. The second coil 28′ is provided by a wound wire (e.g. of copper) which runs around the substrate layers 5, 6, 7 and the two energy stores 9 and 10 of the sensor system 1.

It is also possible only to provide one of these two coils 28, 28′, that is either only the conductor track coil 28 or only the wire coil 28′. These two coils, however, do not differ from one another in their basic functionality and also not from the coil 28 shown in FIG. 3. The wire coil 28′ is fixed within the mold (e.g. by a clamp or by means of suitable abutments in the mold) in the manufacture of the sensor system 1 before the mold is cast with the potting material (for forming the outer skin 2).

The sensor system 1 can be switched on by an external transmitter of a magnetic field (not shown) via a magnetic activation signal using the named reed contact 29 which in this example is arranged on the end piece 11 of the second substrate layer. In the case of a sensor system for fish, the fish can have been previously removed from the water for such a switching-on process to ensure a reliable switching on of the implanted sensor system. The sensor system is preferably configured so that it remains in an activated (switched on) state after a first-time switching on by means of the reed contact 29.

The energy stores 9, 10 of the embodiments shown (FIGS. 1 to 4) can advantageously even be charged via the coils 28 and/or 28′ when the fish with the implanted sensor system 1 is in the water so that therefore the fish does not have to be caught or removed from the water for charging the sensor system 1.

FIG. 5 shows a schematic representation of a stack 31 of thinned substrate layers of the kind proposed here. The stack 31 comprises four thinned substrate layers 5, 6, 7, 32 which each have a thickness of around 10 micrometers and wherein the fourth substrate layer 32 which is the bottommost substrate layer of the stack 31 and which can have a width between 1 mm and 10 mm is bent so that a first part region 33 and a second part region 34 of this substrate layer 32 project out of a common main plane E of the stacked substrate layers 5, 6, 7, 32 of the stack 31. This main plane E extends parallel to the remaining non-bent substrate layers 5, 6, 7.

A first sensor 35 is arranged on the first part region 33 and a second sensors 36 is arranged on the second part region 34, that is, for example, functional components of the measuring unit of a sensor system of the kind proposed here. In this example, the two sensors 35 and 36 are each pressure sensors; however, the sensors 35, 36 could also be configured for measuring a temperature, an acceleration, a radiation intensity, a material concentration (for instance of sugar or insulin, for example in the blood), a pH (for example of blood), a moisture, an electrical current, an electrical voltage, an electrical field or a magnetic field (for instance using a Hall probe). Instead of the sensors 35, 36, functional components of a transmission unit and/or of a reception unit could be arranged such as, for instance a (RF) antenna, a photosensitive sensor or an LED.

In this example, the two part regions 33 and 34—and thus also the sensors 35, 36—include an angle of around 90° with one another and respectively with the main plane E. The propagation of pressure waves (in air or in liquids such as blood) can be resolved in space and in time by this arrangement and signal portions which are associated with a specific source such as a heart can be filtered using time of flight differences in order subsequently to subject them to a frequency analysis. It is also possible by this arrangement of the sensors to locate a source of pressure waves.

The stack 31 therefore comprises a measuring unit having the two sensors 35, 36 arranged (integrated) on the fourth substrate layer 32 for measuring measurement values and for generating corresponding measured signals. The stack 31 furthermore comprises a RF transmission unit (its associated switching circuit is not shown here) arranged on the third substrate layer 7 for transmitting signals using the measured signals and comprises a control and evaluation unit (its associated switching circuits are not shown) connected to the measuring unit and the transmission unit and arranged on the first and second substrate layers 5, 6 for processing the measured signals and controlling the transmission unit for the transmission of the transmitted signals. The evaluation unit is in particular configured for filtering, amplifying and subsequent digital/analog conversion of the measured signals of the two sensors 35, 36 and for carrying out the above-described further evaluation of the measured signals.

The four substrate layers are additionally connected to one another by means of vias (not shown here). Signal conductors (not shown here) are additionally integrated in the fourth substrate 32 for transmitting the measured signals of the sensors 34, 35 to the control and evaluation unit integrated in the first and second substrate layers 5, 6.

The stack 31 can be used, for example, for a sensor system of the kind described here, such as that described with reference to FIGS. 1 to 4. Accordingly, the two pressure sensors 35, 36 could also be arranged at two mutually oppositely disposed sides of the fourth substrate layer 32. In addition, the stack 31 can be connected to an energy storage unit for the energy supply of the units.

A schematic representation of a detail of a specific embodiment of a sensor system 1 of the kind proposed here having a stack 31 of thinned substrate layers of the kind proposed here is shown in FIG. 6. In this respect, an unfinished state of the system 1 is shown during its manufacture. A part of a bent substrate layer 5 of the stack 31 can be recognized, with a part region 33 of the substrate layer 5 projecting out of a main plane E of the stack 31. A pressure sensor 4, that is a functional component of a measuring unit of the sensor system 1, is arranged on this part region 33. It could just as easily be another one of the initially named sensors instead of the pressure sensor 4. It could in this respect, however, also be a functional component of a transmission unit or of a reception unit of the sensor system 1, such as an antenna, a photosensitive sensor or an LED.

The stack 31 on which switching circuits of a control and evaluation unit and of the transmission unit are additionally integrated (not shown) is fixed in a mold 37 which is cast in a following step using a biocompatible potting material (not shown) for completing the system 1. In this state, the part region 33 of the substrate layer 5 is supported at the mold 37 and its position is hereby stabilized by it so that it cannot move back into the main plane E. The mold thus acts as a holding element for fixing and holding the part region 33. In this respect, the pressure sensor 4 is arranged in a passage opening 38 of the mold 37 so that the pressure sensor 4 is also in direct contact with an outer space of the sensor system 1 after casting the mold 37.

After filling the mold 37 with the potting material and after its hardening, the mold 37 is removed. The potting compound then also simultaneously serves as a holding element for fixing the part region 33 in its current position.

### REFERENCE NUMERAL LIST

1 sensor system
2 outer skin
3 electrode
4 pressure sensor
5 first thinned substrate
6 second thinned substrate
7 third thinned substrate
8 via
9 first energy store
10 second energy store
11 end piece of a thinned substrate layer
12 inner space of the system
13 switching circuit for pressure measurement
14 switching circuit for temperature measurement
14′ temperature sensor
15 switching circuit for acceleration measurement
15′ accelerometer
16 switching circuit for measuring a skin resistance and a skin potential
17 switching circuit of the evaluation and control unit
18 switching circuit of the transmission unit
19 intermediate layer
20 circuit of the voltage adaptation circuit and of the recharging circuit
21 contact
22 anode of the first energy store
23 cathode of the first energy store
24 anode of the second energy store
25 cathode of the second energy store
26 electrolyte of the first energy store
27 electrolyte of the second energy store
28 coil
28′ further coil
29 reed contact
30 electrical connector
31 stack of thinned substrate layers
32 fourth thinned substrate layer
33 first part region of the substrate layer
34 second part region of the substrate layer
35 first sensor on the part region
36 second sensor on the part region
37 mold
38 passage opening
E main plane of the stack of the thinned substrate layers

1. A sensor system for implanting into a body of a living being comprising a measuring unit for measuring parameters of the body and for generating corresponding measured signals; a transmission unit for transmitting signals using the measured signals; a control and evaluation unit connected to the measuring unit and the transmission unit for processing the measured signals and controlling the transmission unit for the transmission of the transmission signals; an energy storage unit for the energy supply of the units; and an outer skin which at least partly surrounds the measuring unit, the transmission unit, the evaluation unit and the energy storage unit,
wherein the system comprises at least two thinned substrate layers on which switching circuits of the measuring unit, of the control and evaluation unit and of the transmission unit are integrated, with the substrate layers being stacked over one another and being connected to one another by electrical vias for the signal transmission between the substrate layers.

2. The system of claim 1, wherein the transmission unit is arranged on a first one of the substrate layers; and in that an intermediate layer screening the transmission signals of the transmission unit is arranged between the first substrate layer and a further one of the substrate layers.

3. The system of claim 1, wherein the measuring unit comprises a temperature sensor for measuring a body temperature.

4. The system of claim 1, wherein the measuring unit comprises an accelerometer for measuring an acceleration of the body.

5. The system of claim 1, wherein at least one of the substrate layers is bent so that at least one part region of this at least one substrate layer projects out of a common main plane of the stacked substrate layers, with at least one functional component of the system being placed on this at least one part region, preferably a sensor of the measuring unit, a component of the transmission unit and/or a component of a reception unit of the system for transmitting or receiving signals.

6. The system of claim 5, wherein the at least one part region includes an angle of at least 20° with the common main plane of the substrate layers.

7. The system of claim 5, wherein the at least one sensor placed on the at least one part region projecting out of the common main plane of the substrate layers is configured for measuring a pressure, a temperature, an acceleration, a radiation intensity, a material concentration, a pH, a current, a voltage, an electrical field or a magnetic field.

8. The system of claim 1, wherein the outer skin his elongated so that a length of the outer skin is preferably larger by a factor 2 to 20 than a width and a height of the outer skin.

9. The system of claim 1, wherein the outer skin his produced from a biocompatible potting material, preferably from a potting material containing silicone.

10. The system of claim 1, wherein the measuring unit comprises at least one pressure sensor which is connected to the switching circuit of the measuring unit and which is arranged at an outer side of the outer skin for measuring a pressure in the body.

11. The system of claim 10, wherein a first pressure sensor and a second pressure sensor are arranged at mutually opposite sides of the outer skin.

12. The system of claim 11, wherein the evaluation unit is configured for determining a frequency spectrum of measured signals generated by the at least one pressure sensor and for determining a blood pressure, an environmental pressure, a heart rate and/or a breathing rate of the body from the frequency spectrum.

13. The system of claim 1, wherein the measuring unit comprises at least two electrodes which are connected to the switching circuit of the measuring unit and which are arranged on an outer surface of the outer skin for measuring a skin potential and/or a skin resistance, with at least two of the electrodes preferably running around the outer skin in ring form.

14. The system of claim 1, wherein the energy storage unit comprises at least one first and one second electrical energy store, with the first energy store having a predefined negative anode potential and the second energy store having a predefined positive cathode potential.

15. The system of claim 1, wherein the energy storage unit comprises at least one first and one second plate-like energy store which are oriented parallel to one another.

16. The system of claim 14, wherein an end piece of one of the substrate layers is arranged between the at least one first and second energy stores, with a voltage adaptation circuit furthermore being integrated on this substrate layer, preferably on the named end piece, for adapting a supply voltage for the measuring unit, for the evaluation unit and/or for the transmission unit.

17. The system of claim 1, wherein the energy storage unit comprises a coil for the contactless charging of the energy storage unit by means of magnetic energy.

18. A method for manufacturing a system from claim 1, comprising the following steps:
integrating switching circuits of the measuring unit, of the evaluation and control unit and of the transmission unit on thinned substrate layers;
stacking the substrate layers;
connecting the substrate layers by means of vias;
connecting the energy storage unit to the measuring unit, to the evaluation and control unit and/or to the transmission unit;
fixing the measuring unit, the evaluation and control unit, the transmission unit and the energy storage unit within a mold; and
casting the mold with a potting material.

19. The method of claim 18 which additionally includes at least one of the following further steps:
connecting electrodes for measuring a skin resistance using the switching circuit of the measuring unit integrated on one of the substrate layers and fixing the electrodes to an inner surface of the mold;
connecting at least one pressure sensor to the switching circuit of the measuring unit integrated on one of the substrate layers and fixing the at least one pressure sensor at the inner surface of the mold;
bending at least one of the substrate layers so that at least one part region of this at least one substrate layer projects out of a common main plane of the stacked substrate layers;
placing at least one functional component of the system on this at least one part region of the at least one bent substrate layer projecting out of the common main plane, in particular a sensor of the measuring unit, a functional component of a transmission unit and/or a reception unit for transmitting or receiving signals.
